(19) United States
(12) Patent Application Publication
(10) Pub. No.: US 2023/0001056 A1
(43) Pub. Date: Jan. 5, 2023

(54) ADAPTIVE CHEMICAL POST-PROCESSING OF NONWOVENS FOR CARDIOVASCULAR APPLICATIONS

(71) Applicant: CORTRONIK GMBH, Rostock (DE)

(72) Inventors: Stefan Oschatz, Rostock (DE); Sabine Illner, Rostock (DE); Jonathan Ortelt, Rostock (DE); Daniela Arbeiter, Rostock (DE); Michael Teske, Rostock (DE); Heinz Mueller, Rostock (DE); Niels Grabow, Rostock (DE); Carsten Momma, Rostock (DE); Klaus-Peter Schmitz, Rostock (DE)

(21) Appl. No.: 17/781,084

(22) PCT Filed: Nov. 27, 2020
(86) PCT No.: PCT/EP2020/083711

§ 371 Date: May 31, 2022

(30) Foreign Application Priority Data
Dec. 3, 2019 (DE) 10 2019 132 800.4
Mar. 31, 2020 (EP) 20167129.4

(51) Int. Cl. A61L 31/06; D04H 1/728; D06M 10/10

(52) U.S. Cl. CPC A61L 31/06; D04H 1/728; D06M 10/10; D06M 2101/38

(57) ABSTRACT

A material includes nonwoven fibers and a surface modification that crosslinks the nonwoven fibers together. The surface modification can include chemical reactive groups. The reactive groups can be selected from diisocyanates, alcohols, epoxides, imides, amides, imines, amines, diacrylates, disiloxanes and disilazanes. A method of forming the material electrospins fiber material in the form of fibers into a nonwoven material. A surface modification is introduced to the fibers either by modifying the fiber material before the electrospinning or by modifying the fiber surface after the electrospinning. The fibers are crosslinked to form the crosslinked nonwoven material.

### PRIORITY CLAIM

This application is a 35 U.S.C. 371 US National Phase and claims priority under 35 U.S.C. § 119, 35 U.S.C. 365(b) and all applicable statutes and treaties from prior PCT Application PCT/EP2020/083711, which was filed Nov. 27, 2020, which application claimed priority from German Application Serial Number 10 2019 132 800.4, which was filed Dec. 3, 2019, and from European Application Serial Number 20167129.4, which was filed Mar. 31, 2020.

### FIELD OF THE INVENTION

A field of the invention concerns electrospun nonwoven materials used in biomedical applications, including biomedical application, such as cardiovascular applications.

### BACKGROUND

Electrospun nanofiber nonwovens formed from medically approved permanent or degradable polymers are known, for example as patch structures, graft systems or sheathings of supporting structures for innovative implant modification in the field of medical technology. The possibility of generating nanofiber nonwovens from a variety of polymers by electrospinning processes combines the mechanical robustness of randomly interwoven fibers with the chemical-biological properties of the underlying plastics. In particular, the possibility of processing in controlled processes is an immense advantage over the use of biogenic materials, such as pericardium, for example in the production of heart valve replacement materials. In addition, active substances can be incorporated into the synthetically produced nonwoven structures as well as into film-like coatings, thus generating drug delivery systems. The coating of vascular supports (stents) with nonwoven structures to create sealing stent grafts is well established; commercially available examples are the following: Papyrus (Biotronik), Jostent Graft-Master (Abbott) and Bioweb (Zeus).

Furthermore, it has already been described that materials for the prevention of paravalvular leakages are producible By a combination and subsequent mechanical interlacing of a number of special fibers.

US 2015/0297372 A1 (Abbott Cardiovascular Systems Inc), US 2013/0150943 A1 (Elixir Medical Corp), EP 2 796 112 A1 (Elixir Medical Corp), EP 2 104 521 B1 (Medtronic Vascular Inc) and WO 2005/034806 A1 (Scimed Life Systems Inc) include degradable stents consisting of a polymer or metal main body, which may also have a degradable coating. However, the stent systems do not include a cover modified by post-processing. The patent specifications EP 2 596 765 B1 (Kyoto Medical Planning Co., Ltd) and EP 1 919 532 B1 (Boston Scientific Corp) describe covered stent systems, in which only the cover or the stent scaffold is degradable. A degradable stent scaffold with a permanent cover is also described by WO 2004/016192 A1 (Scimed Life Systems Inc). In this case, however, the cover is located within the stent scaffold, the function of which is mainly to prevent the implant from dislocating in the vessel. A system in which the cover and stent scaffold are degradable is described by WO 2010/117538 A1 (Medtronic Vascular Inc). In addition, EP 2 380 526 A2 describes an implant and a method for its production, which implant includes a covered stent, which may also contain a degradable cover.

At present, a range of products are available that contain nanofiber nonwovens that can be obtained from biocompatible polymers by electrospinning processes. However, without chemical post-treatment, the resulting nonwoven structures are limited to the physico-chemical properties inherent to the polymers used. For example, polyurethane nonwovens show a sudden drop in tensile strength in an aqueous environment, which is due to a splitting of intramolecular hydrogen bonds. Furthermore, currently the fiber thickness (fiber diameter) and the physical bonds between the electrospun fibers are only dependent on the underlying spinning parameters.

The generation of high-performance materials based on electrospun nanofiber nonwovens for implant development is usually based on the use of hybrid materials. These consist of nonwovens of different interwoven fibers formed from different polymers in order to achieve a combination of material properties, including an increase in the tensile strength of the material. Other possibilities include, for example, the spinning of mixtures of different polymers from a single solution, so-called polymer blends, or the application of an additional polymer layer by dipping or spraying processes, although this is accompanied by a loss of the characteristic nonwoven surface morphology.

### SUMMARY OF THE INVENTION

Preferred methods provide for control of the mechanical properties and surface finish of nonwoven materials formed from electrospun fibers. Methods allow post-process chemical modification to adjust the material properties, while retaining the typical nonwoven fiber structure for biomedical applications.

A material includes nonwoven fibers and a surface modification that crosslinks the nonwoven fibers together. The surface modification can include chemical reactive groups. The reactive groups can be selected from diisocyanates, alcohols, epoxides, imides, amides, imines, amines, diacrylates, disiloxanes and disilazanes.

A preferred method of forming the material electrospins fiber material in the form of fibers into a nonwoven material. A surface modification is introduced to the fibers either by modifying the fiber material before the electrospinning or by modifying the fiber surface after the electrospinning, The fibers are crosslinked to form the crosslinked nonwoven material.

### BRIEF DESCRIPTION OF THE DRAWINGS

In the following, embodiments as well as further features and advantages of the invention will be explained by the figures, in which:

FIG. 1 shows a schematic representation of the described post-processing of electrospun (nanofiber) nonwovens; and

FIG. 2 shows a schematic representation of the post-process crosslinking of TSPCU with diisocyanates (A) and an overview of two interpenetrating polymer networks (B); and

FIG. 3 shows representative ATR-IR images of a TSPCU nonwoven as reference material (a) and of TSPCU nonwovens crosslinked with HMDI before thermal post-treatment (b) and after thermal post-treatment (c); and

FIGS. 4A-4D show scanning electron microscope images of diisocyanate-crosslinked TSPCU nonwovens ((4A) HMDI; (4) 4,4′-MBI, (4C) Me3-HMDI and (4D) 4,4′-DMDI), and

FIG. 5 shows an exemplary stress-strain curve of hexamethylene-1,6-diisocyanate-crosslinked TSPCU nonwovens (light, top) compared to unmodified electrospun TSPCU (black, bottom); and

FIG. 6 shows a further stress-strain curve of polyimide nonwovens spun with hexamethylene-1,6-diisocyanate (top) and ethylenediamine (middle) and then thermally treated compared to unmodified electrospun polyimide nonwovens (black, bottom); and

FIGS. 7A-7D show scanning electron microscope images of untreated electrospun TSPCU nanofiber nonwovens (7A-7B) compared with TSPCU nonwovens crosslinked by vapor deposition with HMDI (7C=7D); and

FIG. 8 shows a stress-strain curve of untreated electrospun TSPCU compared to a TSPCU nonwoven crosslinked by vapor deposition with HMDI; and

FIGS. 9A-9D show scanning electron microscope images of electrospun TSPCU nanofiber nonwovens coated with polyallylamine under variation of the process parameters (9A) parameters: 1 min, 1.20 mbar, 60% input (i.e. initial power) and (9B) parameters: 5 min, 0.90 mbar, 60% input (i.e. initial power)) and HMDSO (9C) parameters: 10 min, 0.59 mbar, 10% input (i.e. initial power), (9D) parameters: 5 min, 0.77 mbar, 10% input (i.e. initial power))

FIG. 10 shows the relative stoichiometric composition of TSPCU nonwovens after plasma coating with allylamine (parameters: 1 min, 1.20 mbar, 60% input (i.e. initial power)), and allylamine (parameters: 5 min, 0.90 mbar, 60% input (i.e. initial power)), and HMDSO (parameters: 10 min, 0.59 mbar, 10% input (i.e. initial power)) and HMDSO (parameters: 5 min, 0.77 mbar, 10% input (i.e. initial power)); and

FIG. 11 shows the development of the fiber diameters of TSPCU nonwovens after plasma coating with allylamine (parameters: 1 min, 1.20 mbar, 60% input (i.e. initial power)) and allylamine (parameters: 5 min, 0.90 mbar, 60% input (i.e. initial power)), as well as HMDSO (parameters: 10 min, 0.59 mbar, 10% input (i.e. initial power)) and HMDSO (parameters: 5 min, 0.77 mbar, 10% input (i.e. initial power)) in comparison to an uncoated TSPCU nonwoven; and

FIG. 12 shows mean stress-strain curves of TSPCU nonwovens after plasma coating with allylamine (parameters: 1 min, 1.20 mbar, 60% input (i.e. initial power)) and allylamine (parameters: 5 min, 0.90 mbar, 60% input (i.e. initial power)) and HMDSO (parameters: 10 min, 0.59 mbar, 10% input (i.e. initial power)) and HMDSO (parameters: 5 min, 0.77 mbar, 10% input (i.e. initial power)) in comparison to an uncoated TSPCU nonwoven (the middle line of each depicted type of lines is the mean and the respective lines of same type surrounding each central mean line correspond to the standard deviation (n=6)).

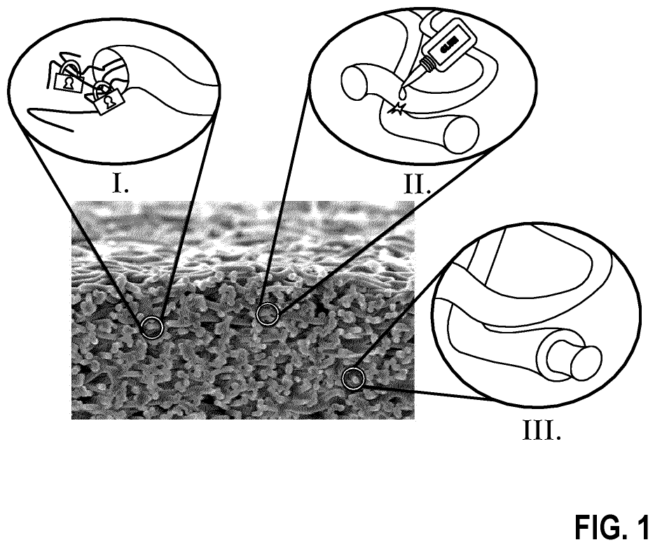

### DETAILED DESCRIPTION OF THE PREFERRED EMBODIMENTS

Nonwoven fiber materials that are crosslinked are provided herein and their use in medical devices is also provided. In methods of forming, post-process adjustment of the physico-chemical properties as well as the tissue-implant reaction of polymer-based implants to specifically defined parameters is achieved for the generation of high-performance materials starting from electrospun nanofiber nonwovens. In particular, the invention can be used for form spatially resolved material modifications while preserving the nonwoven fiber structure, since this filigree fiber structure can be made mainly responsible for the suitability as implant material, such as atrioventricular valve replacement material or sheathing material.

Accordingly, a non-woven material formed from electrospun fibers, in particular nanofibers, is provided which has a surface modification by which the fibers may be crosslinked with each other. In particular, the electrospun fibers are crosslinked. In one embodiment, the surface modification is present in the form of chemical reactive groups. In one embodiment, the fiber material is selected from the group including or consisting of polyurethanes, polyimides, polyamides, polyesters and polytetrafluoroethylenes.

The chemical reactive groups may be selected here from the group including or consisting of diisocyanates, alcohols, epoxides, imides, amides, imines, amines, especially allylamines, diacrylates, disiloxanes and disilazanes.

According to the invention, the introduction of surface modification allows the mechanical properties, in particular tensile strength and plastic elongation range, to be adapted advantageously by subsequently blocking the mobility of the polymer chains via (photo)chemical or thermal crosslinking, which affects surface polarity, in particular with regard to biocompatibility, cell growth and endothelialisation. When using biodegradable materials, which optionally carry an active substance, the speed at which degradation or active substance release processes occur can also be adjusted, which can be achieved by the post-process creation of a diffusion barrier.

The above-mentioned modifications can be carried out with easily realisable process steps, while also preserving the typical fiber structure of the electrospun nonwovens, as these are mainly responsible for the mechanical suitability of nanofiber nonwovens in the cardiovascular area. Therefore, the present invention addresses the possibility of modifying electrospun nanofiber nonwovens while preserving the fiber structure post-process, i.e. after the actual spinning process, by treatment with heat or UV radiation, or by applying thin or ultra-thin layers by vapor deposition or PECVD technologies with respect to their physico-chemical properties in such a way that they meet the requirements of the particular site of use as an implant. This means that nonwoven materials tailored to the site of use can be made accessible in just a few processing steps.

The biocompatible nonwoven materials created in this way open up new fields of application in the development of implants in terms of their material properties. In particular, the fields of application constituted by stents, TAVI procedures, and coatings for implants, for example for pacemaker and implantable defibrillator electrodes, are addressed.

In one embodiment, a nonwoven material formed from electrospun fibers, in particular nanofibers, is provided, having a surface modification by which the fibers can be crosslinked with one another, characterised in that the fiber material is a polyimide and the surface modification is in the form of diisocyanates or amines, in particular diamines.

Such an embodiment has the advantage that delamination problems can be eliminated. This is because one technical problem is the tendency of some polymers, such as polyimide, to delaminate too much after they have been spun into nonwovens. More precisely, there is insufficient physical crosslinking (bonding) between two or more fibers (unbonded fiber fabrics). This can be counteracted by the subsequent application of a fixation layer by (plasma-activated) vaporisation or spraying processes. According to the invention, this layer consists of reactive, volatile molecules, for example 1 ,6-diisocyanatohexane (HMDI) or 1,2-ethylene diamine, which precipitates from a saturated gas phase on the surface of the nonwoven and fixes the polymer fibers at the contact points, thus creating a dimensionally stable network.

A further aspect of the present invention is a method for producing a crosslinked electrospun nonwoven material including the steps of:
providing a fiber material,
electrospinning the fiber material in the form of fibers into a nonwoven material,
introducing a surface modification to the fibers either by modifying the fiber material before the electrospinning or by modifying the fiber surface after the electrospinning,
crosslinking the fibers to form a crosslinked nonwoven material.

In one embodiment, it is provided that the introduction of a surface modification is carried out by adding a reactive chemical substance to the fiber material before the electrospinning. In one embodiment, the surface modification is achieved by adding diisocyanates, alcohols, epoxides, imides, amides, imines, amines, in particular allylamines, diacrylates, disiloxanes and disilazanes.

In one embodiment, the active chemical groups are added to the polymer already before the spinning process. In subsequent post-processing treatment, among other things, the loss of tensile strength in the medium can be compensated for by increasing the tensile strength overall.

By introducing reactive chemical substances during the spinning process, individual nonwoven layers can also be irreversibly bonded together by chemical bonds during post-processing. Therefore, nonwoven-like composite materials can be created with a layered structure (sandwich structure), while preserving the fiber structure. Depending on the choice of the polymers used, these composites exhibit novel physico-chemical properties that differ greatly from the material properties of the individual components. This technology represents an alternative to established contact welding or bonding methods, for example to embed the struts of a stent scaffold in the nonwoven.

By introducing reactive chemical substances, such as diisocyanates or diacrylates, into, for example, thermoplastic silicone polycarbonate-urethanes (TSPCUs) prior to the actual spinning process, the system can be additionally crosslinked at the molecular level through activation processes, such as thermal treatment (annealing) or treatment with UV radiation, while maintaining the same level of processing. This can be used to adjust the material hardness as well as to generate joins.

In a further embodiment of the method provided herein, the introduction of a surface modification is carried out by applying a reactive chemical substance to the fiber material after electrospinning. This can be achieved, for example, by vapor deposition with reactive components using highly volatile reactants. This enables additional chemical as well as physical connections between different fibers of the polymer nonwoven at the fiber surface.

Among other things, a mutual displacement of the individual fibers can be blocked. As the nonwoven fibers still have sufficient mobility after the treatment, the pronounced extensibility of the material can be extended by an increased tensile strength.

In a preferred embodiment, the introduction of a reactive chemical substance to the fiber material is carried out at elevated temperature, especially in the range of 50 to 90° C. In this temperature range, the morphology of the nonwoven remains unchanged, while it is possible to transfer a high concentration of the reactive chemical substance to the nonwoven fibers, which allows a strong crosslinking of the nonwoven fibers. In another embodiment of this process step, the introduction is performed at an elevated temperature in the presence of water, especially in the presence of (water) vapor.

Alternatively, there is the possibility of creating a barrier layer as a diffusion barrier by plasma-chemical application of ultra-thin polymer layers via plasma polymerisation (PECVD, plasma-enhanced chemical vapor deposition), which, for example, prevents hydration of urethane groups. The advantage of this process lies in the low thermal load on the nanofiber nonwoven structures, as the reaction energy is supplied by a plasma instead of by temperature, and the homogeneous layer formation in the nanometre range. Depending on the choice of the precursor monomers in the gas space of the reaction chamber, this layer can be biostable or biodegradable and thus may have an effect on the degradation behaviour of biodegradable nonwovens. In addition, this layer can modulate the release of active substances incorporated in the nonwoven and may significantly reduce a burst release.

The additional chemical post-processing advantageously makes it possible to individually adjust and increase the morphology of the obtained nonwovens, for example the pore size and the mechanical strength, especially tensile strength. This post-processing refers to a chemical crosslinking and thus to the formation of covalent bonds between the molecules. A chemical crosslinking of molecule chains as well as individual nonwoven fibers at their contact points is particularly successful when using, for example, diisocyanates or diacrylates as suggested herein.

Especially by post-treatment of electrospun nanofiber nonwovens by plasma deposition processes (PECVD/Plasma Enhanced Chemical Vapor Deposition) ultra-thin layers can be generated while preserving the structure of the nonwoven fibers. Depending on the choice of starting materials for PECVD, these precursor monomers can change the mechanical properties, cause a change in surface polarity, or act as a permanent or biodegradable diffusion barrier, which ultimately has an effect on drug release and degradation processes.

As already mentioned, the crosslinking of the fibers with each other to form a crosslinked nonwoven material can be carried out by thermal post-treatment or by irradiating reactive light, especially UV light.

Further advantages are the modification of the surface morphology and the fiber diameters while preserving or modifying the mechanical properties, the guarantee of the shelf life of the materials with incorporated reactive molecules to allow processing in several process steps, and the use of nonwovens with reactive species embedded in the fibers for seamless joining of layered nonwoven materials.

By introducing reactive chemical substances such as diisocyanates or diacrylates, polymers can be adjusted covalently, by forming allophanate groups for example, or physically, by generating interpenetrating networks, in respect of their mechanical properties as well as with regard to their behaviour in the medium and their tendency to delamination. In addition, tear-resistant joins can be produced. Due to the given shelf life of the electrospun nanofiber nonwovens with incorporated reactive species, a further downstream processing of the materials is possible while preserving the intrinsic reactivity. The processes therefore can be embedded in a staggered process chain.

It should be emphasised that the characteristic fiber structure of the nanofiber nonwoven is retained in the post-process modifications described in accordance with the invention. By the methods described, adaptively electrospun nonwoven materials can be adjusted with regard to their physico-chemical properties, such as mechanics, surface polarity and finish, while preserving the same fiber structure. However, according to the invention, it is also possible to post-process the nonwoven morphology, such as surface structure and packing density, while maintaining the same mechanical properties.

In one embodiment, it is provided that a surface modification is applied to the fibers by applying at least one thin nano-layer, preferably in a thickness in the range of 400 to 600 nm.

By applying ultra-thin nano-layers (smaller than 2 μm, preferably smaller than 1 μm), for example by PECVD, it is also possible to create barrier layers which affect the interactions of the material with ambient media or to form diffusion barriers through these layers, which can ultimately have an effect on drug release processes and the degradation behaviour of the polymer materials.

Furthermore, the technology according to the invention opens up the possibility of applying polymeric layers by vapor deposition processes or ultra-thin polymeric layers by PECVD, which can fix the fibers while preserving the structure and, depending on the choice of the applied layer, may control the surface polarity or diffusion. The biological reaction as well as the degradation behaviour and the release rate of active substances incorporated in the nonwoven thus can be modulated.

The possibility of post-process modification of electrospun nanofiber nonwovens opens up a broad field of biomedical applications, among other things because the physico-chemical properties of a nonwoven material can be subsequently improved while preserving its morphology and can be adapted flexibly and, in part, spatially resolved to suit specific requirements.

Therefore, one aspect of the present invention is to use the nonwoven materials provided herein as a component for medical devices, in particular medical implants. Accordingly, a further aspect of the present invention aims to provide a medical implant including a non-woven material provided herein.

Such a medical implant can be a stent, for example in the form of a “covered” stent (stent graft), or a heart valve prosthesis and is particularly suitable for cardiovascular applications.

### PRACTICAL EXAMPLES

### Example 1

The polymer solution used for electrospinning, for example 7.5 wt. % thermoplastic silicone polycarbonate-urethane (TSPCU) in chloroform (CHCl3), trifluoroethanol (TFE) and dimethylformamide (DMF), is supplemented by reactive components added in a ratio of 1 to 50% relative to the polymer mass, and the mixture is spun within 6 hours. These reactive components include diisocyanates, such as hexamethylene diisocyanate (HMDI), methylene-(bisphenyl-isocyanate) or lysine diisocyanate. The functional groups of the isocyanates are retained during the spinning process and are homogeneously distributed in the polymer fibers. The reaction of the isocyanates with the provided functional groups of the polymer, such as urethane, amide, amine or alcohol groups, is stimulated by thermal treatment of the nonwoven thus obtained, and a chemical crosslinking process takes place both within the fibers and at the contact points of individual fibers, so that the fibers are covalently bonded to each other and can no longer be displaced relative to each other. This results in a reduced tendency to delamination and increased tensile strength of the nonwoven compared to the untreated material.

In particular, it has been shown that nonwovens with incorporated diisocyanates obtained in this way can be stored at −20° C. without losing the reactive components. This is interesting in that a processing with a number of individual process steps can be added. This technology can be used to embed structures, for example a stent, into the nonwoven scaffold without risking a separation of the fibers at the insertion point. This allows seamless joining, which results in a much higher tearing strength.

The incorporation of reactive components into the spinning process can also be extended to other electrospun polymers and is not limited to TSPCU. For example, the incorporation of HMDI or allylamine during the spinning process of polyimide with subsequent thermal post-treatment results in a strong increase of the tensile strength or stabilisation of the nonwoven compared to delamination.

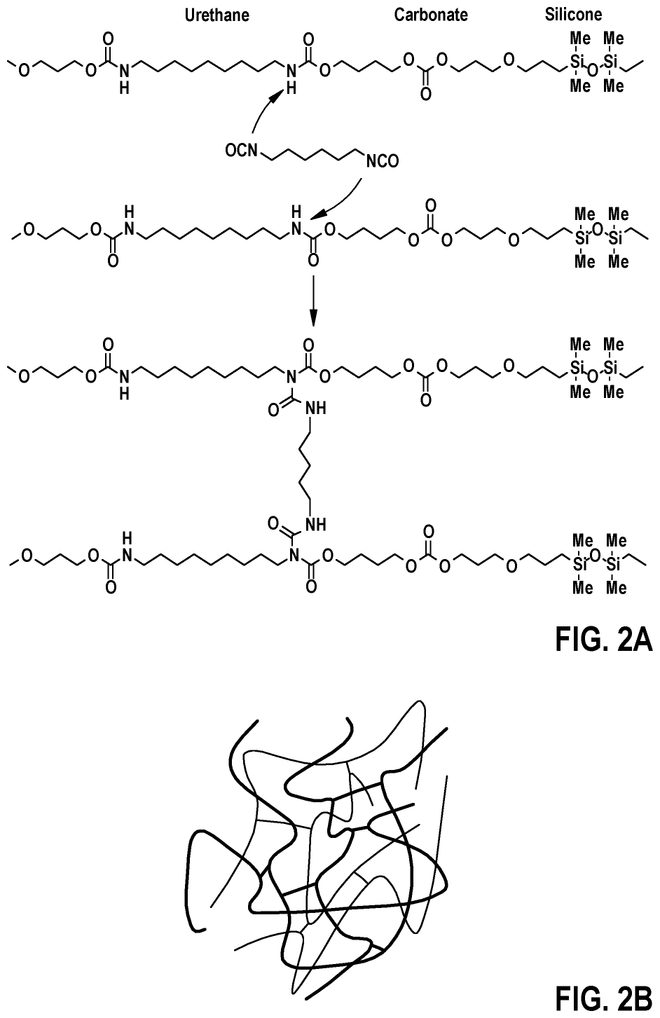

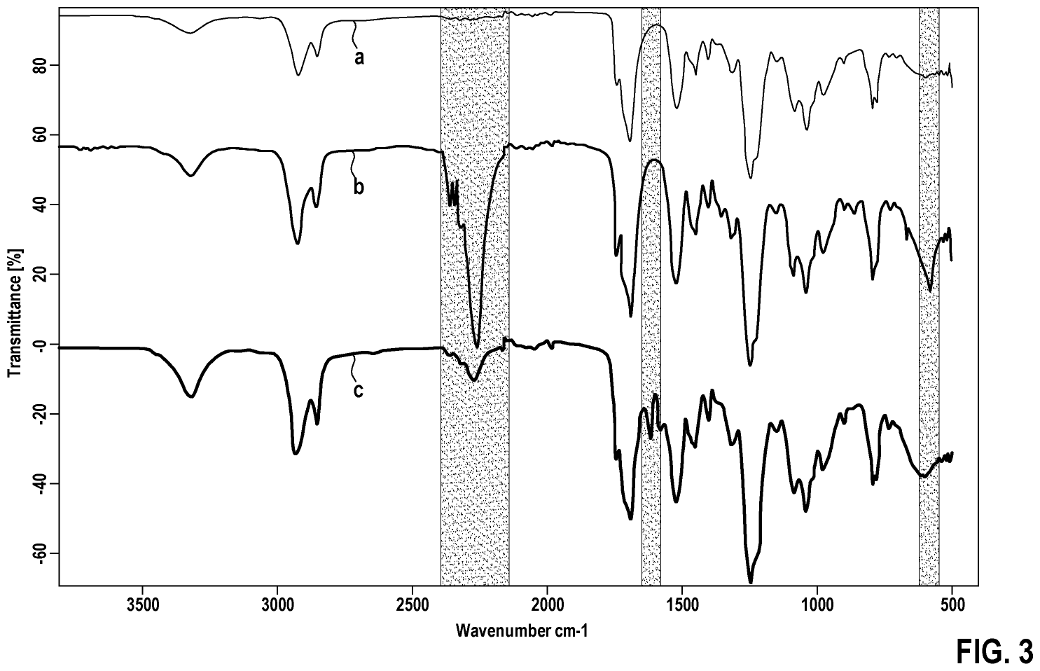

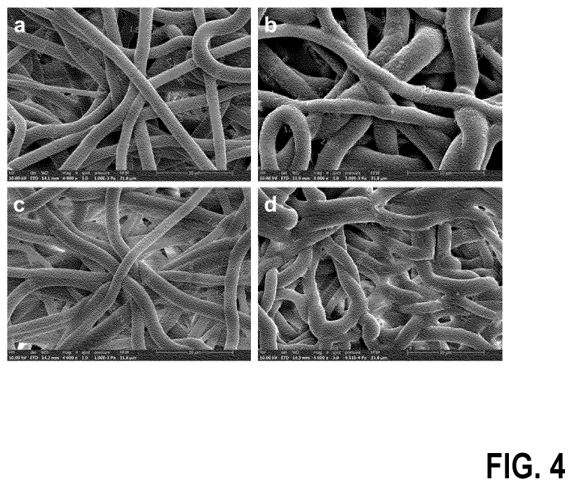

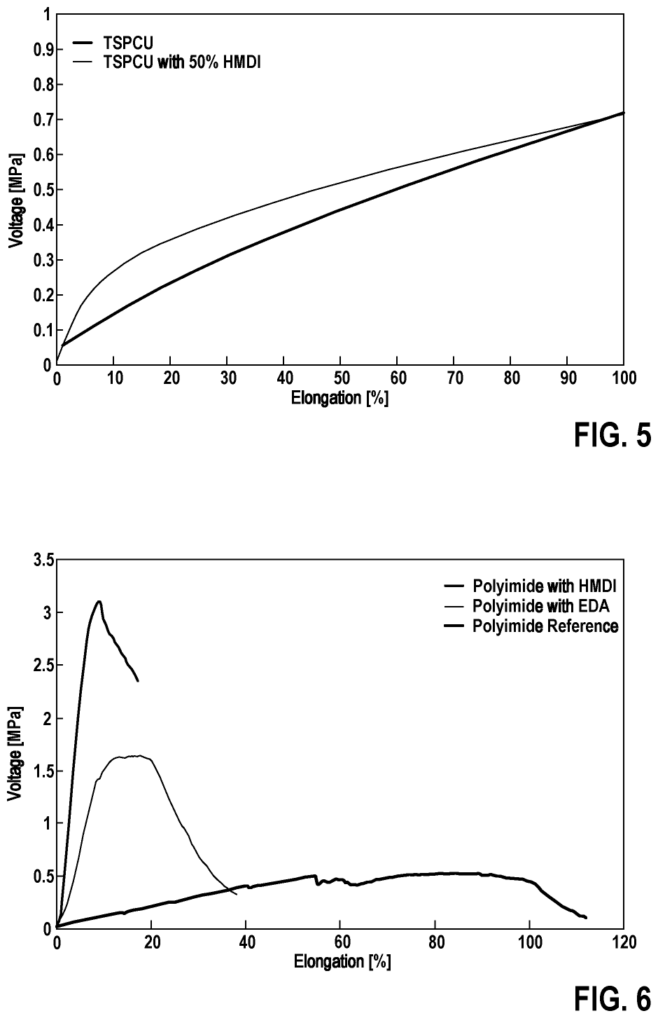

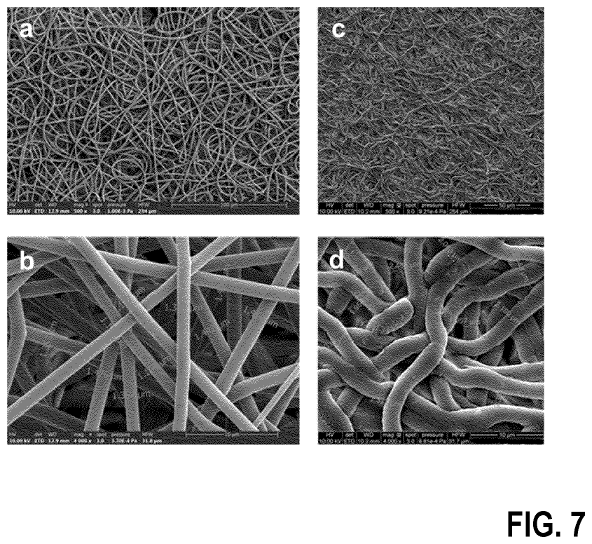

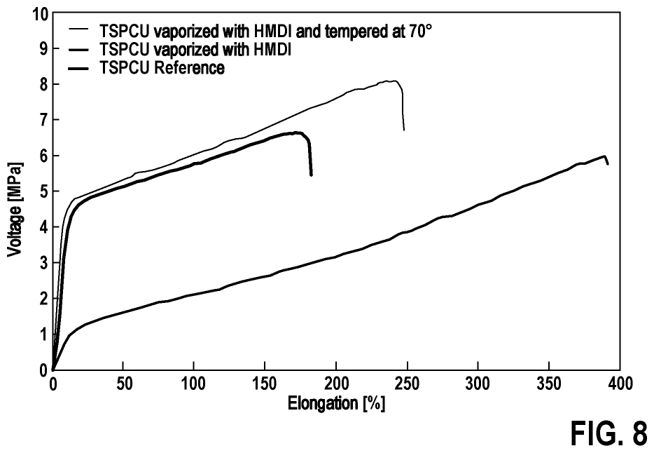

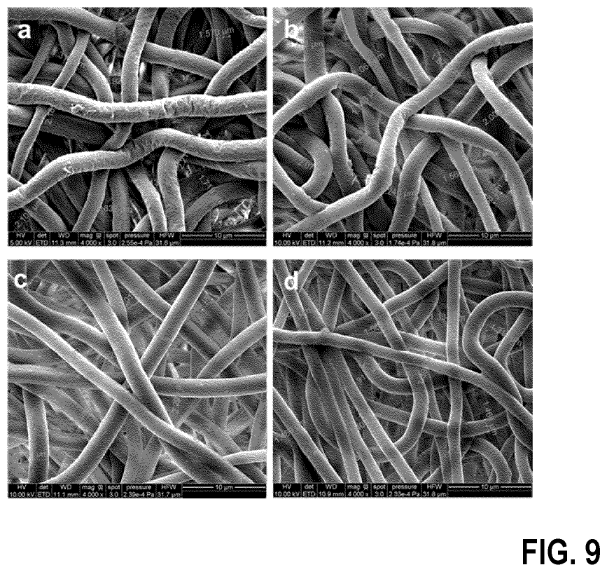

In addition, a purely physical crosslinking of the individual polymer chains can also take place in the absence of sufficient reactive groups, by forming an interpenetrating network (see FIG. 2 II), based on the diisocyanates. A further possibility of forming such an interpenetrating network is the use of diacrylates in the spinning process and subsequent photoinduced crosslinking of these acrylates. In this way, the polymer chains of the electrospun material are intertwined with oligoacrylates. According to the invention, the macroscopic structure of the nonwoven is maintained, with a simultaneous change in the mechanical stability of the material.

### Example 2

By vapor deposition of electrospun nanofiber nonwovens by reactive species such as volatile diisocyanates, layers can be applied to the individual fibers, which on the one hand fix them against displacement of the nonwoven fibers among themselves and on the other hand can shield them against solvent effects, such as hydrating of hydrogen bonds. For post-processing, the nonwoven material is incubated at room temperature and 70° C. in a saturated steam atmosphere of diisocyanate to achieve surface crosslinking. The fiber structure of the material is preserved by the gentle vaporisation process.

Depending on the choice of vaporising materials, as well as the temperature and duration of the process, the thickness of the layer applied to the nonwoven can be modified according to the requirements. In addition, the effect of delamination of electrospun nonwovens can be minimised by this process, as the fibers crosslink with each other on a macroscopic level. By the described post-processing, materials are obtained which have a modified fiber morphology and fiber surface as well as increased packing density of the fibers while preserving the mechanical properties.

### Example 3

A post-process application of ultra-thin layers in the range of 1-2 μm to the fibers of the nanofiber nonwoven by Plasma Enhanced Chemical Vapor Deposition (PECVD) is also described in accordance with the invention. Here, monomers excited by low-pressure plasma are made to polymerise on the nonwoven surface and form polymer layers on the fibers while preserving the fiber morphology. This process is particularly impressive because of the wide range of monomers that can be used for the application process. This makes it possible to fix the nonwoven fibers against each other in a similar way to a vaporisation process or to shield them from the surrounding medium by the applied layer. The low thickness of the applied layers is remarkable. Furthermore, by using degradable starting materials, e.g. acrylate-functionalised esters, a degradable layer can be applied to the individual fibers, which modifies degradation and release processes. Depending on the choice of the monomer used, these layers also have a high ductility, which is particularly positive in respect of their use as a coating technology for strongly moving parts, such as those found in a TAVI or stent. By selecting the process parameters of the PECVD, the applied coatings can be modified in their stoichiometry as well as their structural composition, such as coating thickness. The described technique of applying ultra-thin layers by PECVD can be used for both biostable and biodegradable polymers and is not limited to certain classes of polymers.

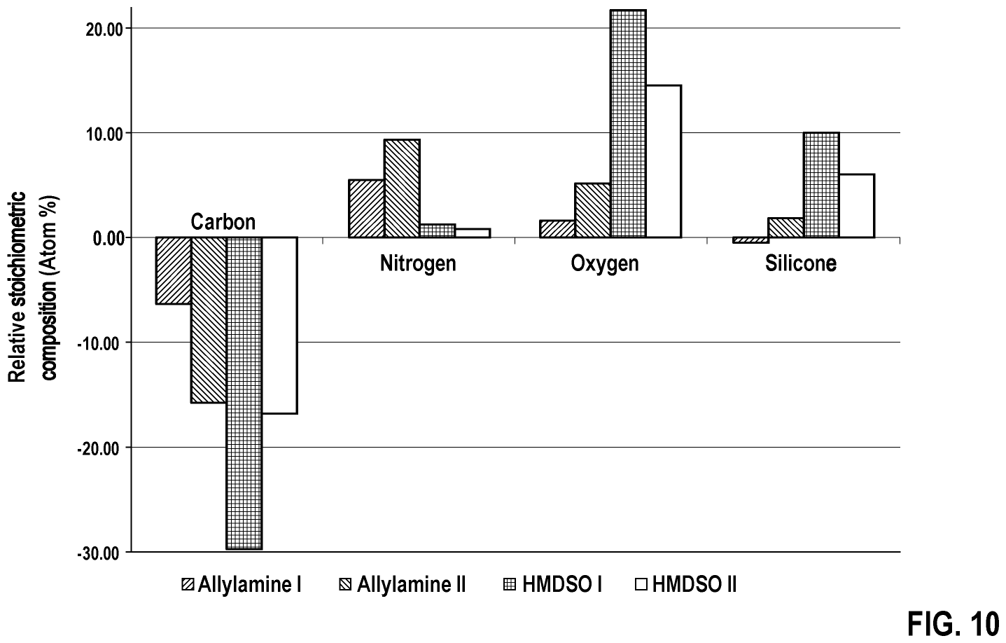

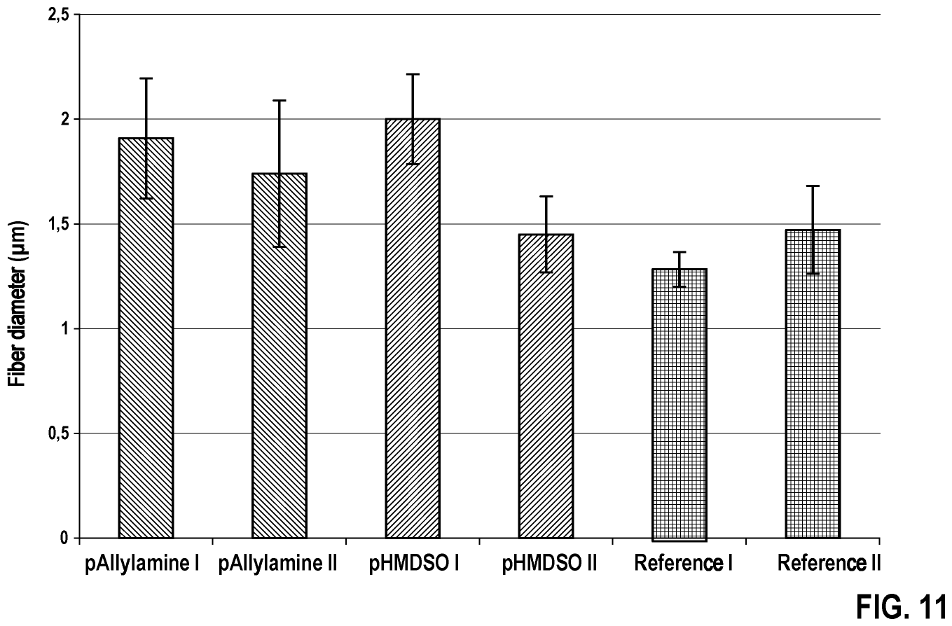

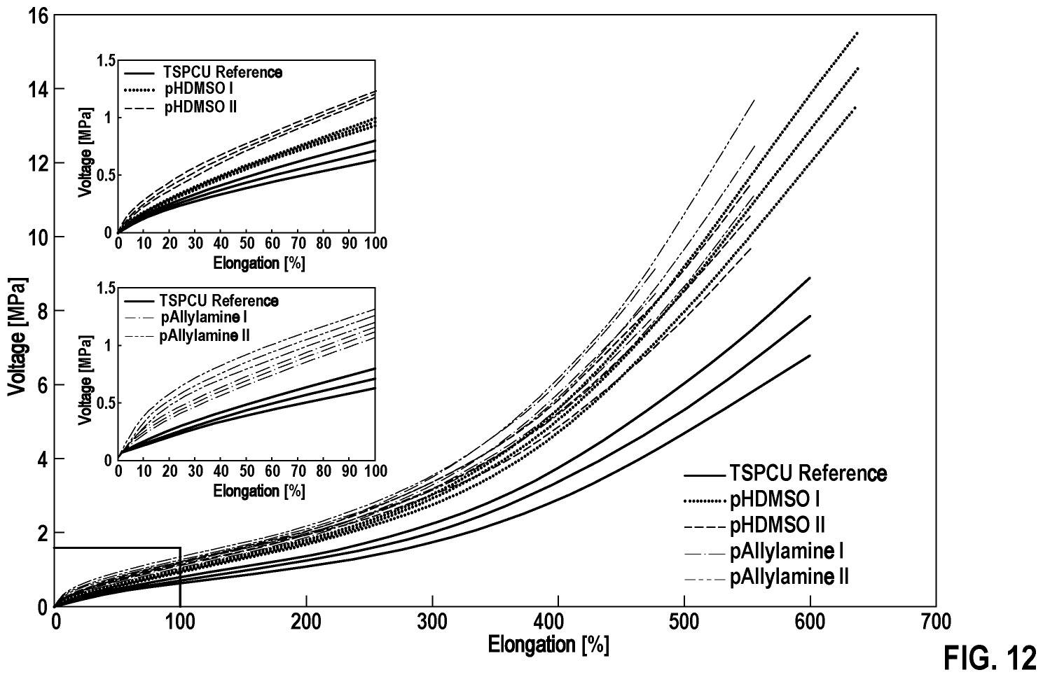

The change in the stoichiometry when applying ultra-thin layers of p-allylamine -and p-hexamethyldisiloxane (HDMSO) compared to untreated TSPCU is shown in FIG. 10 as a differential representation. The percentage decrease in carbon can be seen with a simultaneous increase in the nitrogen or silicon content.

It will be apparent to those skilled in the art that numerous modifications and variations of the described examples and embodiments are possible in light of the above teaching. The disclosed examples and embodiments are presented for purposes of illustration only. Therefore, it is the intent to cover all such modifications and alternate embodiments as may come within the true scope of this invention.

1. A material comprising non woven fibers and a surface modification that crosslinks the nonwoven fiber together.

2. (canceled)

3. The material according to claim 1, wherein the surface modification comprises crosslinking chemical reactive groups.

4. The material according to claim 3, wherein the chemical reactive groups are selected from the group comprising or consisting of diisocyanates, alcohols, epoxides, imides, amides, imines, amines, diacrylates, disiloxanes and disilazanes.

5. The material according to claim 1, wherein the fibers are formed of fiber material selected from the group comprising or consisting of polyurethanes, polyimides, polyamides, polyesters and polytetrafluoroethylenes.

6. The material according to claim 5, wherein the fiber material is thermoplastic silicone polycarbonate-urethane.

7. A method for producing a crosslinked electrospun nonwoven material comprising the steps of:
providing a film material,
electrospinning the fiber material in the form of fibers into a nonwoven material,
introducing a surface modification to the fibers either by modifying the fiber material before the electrospinning or by modifying the fiber surface after the electrospinning,
crosslinking the fibers to form a crosslinked nonwoven material.

8. The method according to claim 7, wherein the introducing comprises adding a reactive chemical substance to the fiber material before the electrospinning.

9. The method according to claim 7, wherein the introducing comprises applying a reactive chemical substance to the fiber material after the electrospinning.

10. The method according to claim 9, wherein the applying comprises vapor depositing the reactive chemical substance on the fiber material.

11. The method according to claim 7, wherein the applying comprising depositing a polymer on surfaces of the fibers by plasma-assisted chemical vapor deposition.

12. (canceled)

13. A medical implant comprising a material according to claim 1.

14. The material of claim 4, wherein the amines are allylamines

15. The method according to claim 10, wherein the vapor-depositing comprises plasma assisted chemical vapor deposition.
